(19) United States
(12) Patent Application Publication
(10) Pub. No.: US 2016/0199367 A1
(43) Pub. Date: Jul. 14, 2016

(54) Composition for the prevention or treatment of the symptoms in the stroke comprising the inhibitor of Pin1

(71) Applicant: RESEARCH & BUSINESS FOUNDATION SUNGKYUNKWAN UNIVERSITY, Suwon-si (KR)

(72) Inventors: Dong Gyu JO, Suwon-si (KR); Sang-Ha BAIK, Suwon-si (KR)

(73) Assignee: RESEARCH & BUSINESS FOUNDATION SUNGKYUNKWAN UNIVERSITY, Suwon-si (KR)

(21) Appl. No.: 14/827,465

(22) Filed: Aug. 17, 2015

(30) Foreign Application Priority Data
Jan. 12, 2015 (KR) 10-2015-0004047

(51) Int. Cl. A61K 31/4745; A61K 31/27; A61K 31/122

(52) U.S. Cl. CPC A61K 31/4745; A61K 31/122; A61K 31/27

(57) ABSTRACT

Provided is a composition for preventing or treating stroke, and more particularly, a pharmaceutical composition for preventing or treating stroke, which contains a peptidyl-prolyl cis-trans isomerase NIMA-interacting 1 (Pin1) inhibitor as an active ingredient.

The Pin1 inhibitor has a neuroprotective effect in which production of NICD1, which is a Notch1 signaling activation product activated in ischemic stroke, is regulated, and cerebral infarction and deficits of neuronal cells triggered by ischemic stroke and neuronal cell death are reduced, and thus can be useful in preventing or treating stroke and also useful in treating a cerebrovascular disease and stroke-associated dementia.

### CROSS-REFERENCE TO RELATED APPLICATION

This application claims priority to and the benefit of Korean Patent Application No. 10-2015-0004047, filed on Jan. 12, 2015, the disclosure of which is incorporated herein by reference in its entirety.

The present invention was undertaken with the support of Mid-career Researcher Program No. NRF-2015R1A2A1A01003530 grant funded by the Ministry of Science, ICT and future planning and Disease Overcoming Technology Development Project No. HI14C2539000014 grant funded by the Ministry of Health and Welfare.

### TECHNICAL FIELD

The present invention relates to a composition for preventing or treating stroke, and more particularly, to a pharmaceutical composition for preventing or treating stroke, which contains a peptidyl-prolyl cis-trans isomerase NIMA-interacting 1 (Pin1) inhibitor as an active ingredient.

### BACKGROUND ART

Stroke is the common name for all symptoms of regional neurological deficits suddenly triggered by cerebral blood flow abnormalities, and is known as a cerebrovascular accident in medical terminology. Stroke is classified into ischemic stroke triggered by blockage of blood vessels supplying blood to the brain and cerebral hemorrhage triggered by bursting of blood vessels in the brain.

Ischemic stroke may be classified into two subtypes: intracranial atherosclerosis, which is blockage of blood vessels in the brain by a narrowing inner diameter of the blood vessels due to the deposit of cholesterol to the wall thereof; and cerebral embolism, which is interruption of cerebral blood flow by a blood clot separated from an artery of the neck or heart. Ischemic stroke is also classified into complete ischemia and incomplete ischemia depending on a circulatory disorder, and in complete ischemia, cerebral infarction, which is a phenomenon of the death of brain cells due to complete blockage of blood circulation in a topical region of the brain, occurs. The brain takes up 20% of the oxygen consumption of the entire body and only uses glucose as an energy source. Therefore, when energy supply is interrupted even for a moment by the blockage of the blood flow, the necrosis of brain cells easily occurs, and a disorder caused by the necrosis of the brain cells, which is the consequence of the cerebral infarction, remains permanently. The most common cause of ischemic stroke is the blockage of cerebral blood flow due to arteriosclerosis occurring in blood vessels supplying blood to the brain due to high blood pressure, diabetes, or hyperlipidemia. Other than that, ischemic stroke is probably triggered by the blockage of cerebral blood flow with blood clots formed from the heart because of sequela such as cardiac arrhythmia, cardiac insufficiency and myocardial infarction, or uncommonly triggered by blood clots formed because of a disease such as Moyamoya syndrome or hyperhomocysteinemia and other body wastes.

Stroke is one of the main causes of death in the world, and according to the World Health Organization statistics for 2008, cerebrovascular disease was the third cause of death, and annually more than 16 million patients appear, and 5 to 6 million patients were listed as having died from stroke. In Korea, based on 2010, the mortality from stroke was 53.2 per 100,000 of population, and thus stroke ranked second behind cancer. Compared to 2000, the mortality from stroke was reduced, but since the occurrence of stroke is also influenced by age, the risk of stroke is expected to continue because of the global trend towards aging population. Stroke is a disease causing a considerable economic burden to both families and society because it remains a serious neurological disorder in many cases even if a patient live through the stroke, and thus rehabilitation and treatment cost a lot of money, and great help from family members or others is needed. Stroke is also known as a main cause of dementia.

The treatment of stroke includes surgical operation for making blood flow smoothly after the risk of the disease is found in an early stage through CT or MRI, acute treatment right after the occurrence of the disease, rehabilitation treatment, and treatment for preventing rehabilitation. Most therapies for ischemic stroke are acute treatments, which include antithrombotic therapies such as a thrombolytic agent for dissolving a blood clot and an antiplatelet agent for preventing the generation of a blood clot. As drugs used in the antithrombolytic therapy, antiplatelet agents such as aspirin, anticoagulants such as heparin and warfarin, and antithrombolytic agents such as a recombinant tissue plasminogen activator (rT-PA) are used. However, the greatest risk of stroke is death or sequela by brain damage, which is caused by the necrosis of brain neuronal cells, in spite of such acute treatments. To prevent the neuronal cell death, the development of a variety of therapeutic agents such as NMDA receptor antagonists, calcium channel brokers, and free-radical scavengers has progressed worldwide, but most of the therapeutic agents were used limitedly or did not pass clinical tests because of side effects such as hallucinations, low blood pressure, edema, cardiac dysfunction, etc. Therefore, it is urgent to develop protective agents for neuronal cells which can prevent the necrosis of the neuronal cells.

Meanwhile, peptidyl-prolyl cis-trans isomerase NIMA-interacting 1 (Pin1) serves as a molecular switch that regulates activation or inactivation of specific proteins by isomerization of a peptidyl group of a substrate into cis or trans. Pin1 is composed of a WW domain that recognizes an amino acid motif of a substrate, for example, phosphorylated serine, threonine or proline, and a PPIase domain that changes a structure of the substrate to a cis or trans type. Pin1 is involved in translocation of specific proteins to a nucleus or mitochondria, regulation of interaction with a target protein, an increase in transcription activity of transcription factor proteins, and an increase in enzyme activity.

Recently, it has been reported that Pin1 plays an important role in the neuronal cell death induced by c-Jun N-terminal kinase (JNK). That is, when Pin1 is inhibited, the neuronal cell death was reduced under oxidative stress or glutamate excitotoxicity. It has been also known that Pin1 interacts with proteins involved in p53 and Notch1 signaling pathways related to the neuronal cell death, or related cell membrane proteins.

Notch is a cell membrane receptor regulating cell division, differentiation and development to a variety of tissues, and there are four types of Notch1 to 4 of mammalian cells. Notch1 is involved in differentiation and maintenance of neural progenitor cells and plays an important role in regulation of a cell cycle and synaptic plasticity. When Notch1 is activated by binding of a ligand, two-step proteolytic degradation occurs due to a metalloprotease, ADAM, and a γ-secretase, and Notch intracellular domain 1 (NICD1) is produced to serve as a transcriptional regulatory factor after the nuclear translocation of NICD1. Recently, research has found that the activation of Notch1 is related to apoptosis, and ischemic conditions of cerebral ischemia induce the activation of Notch1, and thus contribute to induction of neuronal cell death.

The above results showed that Pin1 interacts with Notch1 to play an important role in the neuronal cell death caused by other stresses like ischemic stroke. However, specific research on this has not been reported yet.

### DISCLOSURE

### Technical Problem

To solve the above-described conventional problems, the inventors found that, under the condition of neuronal cell death regulated by Pin1, Pin1 inhibitors including juglone have an effect of protecting neuronal cells in that they lower a signaling activity of Notch1 inducing apoptosis, thereby reducing apoptosis, and inhibit the triggering of cerebral infarction because of middle cerebral artery occlusion in an animal model with an ischemic brain nerve system disease, thus completing the present invention.

Therefore, the present invention is directed to providing a pharmaceutical composition for preventing or treating stroke, which contains a Pin1 inhibitor as an active ingredient.

However, the technical problems to be accomplished by the present invention are not limited to the above-described problems, and other problems not described herein will be fully understood by those of ordinary skill in the art from the following descriptions.

### Technical Solution

To address the issues described above, the present invention provides a pharmaceutical composition for preventing or treating stroke, which contains a Pin1 inhibitor as an active ingredient.

In one example embodiment of the present invention, the stroke is ischemic stroke.

In another example embodiment of the present invention, the inhibitor may be at least one selected from the group consisting of 5-hydroxy-1,4-naphthoquinone (juglone), diethyl-1,3,6,8-tetrahydro-1,3,6,8-tetraoxobenzo phenanthroline-2,7-diacetate (PiB), and dipentamethylene thiuram monosulfide (DTM).

In still another example embodiment of the present invention, the composition inhibits the expression or activity of Pin1.

In yet another example embodiment of the present invention, the composition has a neuroprotective effect by inhibiting the activity of Notch1.

In yet another example embodiment of the present invention, the composition may further contain a pharmaceutically acceptable carrier or additive.

In another aspect, the present invention provides a method of preventing or treating stroke, which includes administering the composition of the present invention to a subject.

In still another aspect, the present invention provides a use of the composition to prevent or treat stroke.

### Advantageous Effects

A Pin1 inhibitor of the present invention has a neuroprotective effect of reducing neuronal cell death by inhibiting the production of a product of the activation of Notch1 signaling, that is, NICD1, activated when ischemic stroke occurs, and reducing cerebral infarction and deficits of neuronal cells caused by ischemic stroke, and thus can be useful in preventing or treating stroke and also useful in treating a cerebrovascular disease and stroke-associated dementia.

### BRIEF DESCRIPTION OF THE DRAWINGS

FIGS. 1A-1C illustrates the experimental results of verifying that Pin1 regulates a Notch1 signaling activity, in which FIG. 1A illustrates the result showing that the expression of an NICD1 protein increases when human Pin1 is overexpressed in a neuroblastoma cell line, SH-SY5Y, FIG. 1B illustrates the result showing that the expression of the NICD1 protein does not increase when a γ-secretase inhibitor (DAPT) is treated after the overexpression of Pin1, and FIG. 1C illustrates the result showing that the expression of the NICD1 protein decreases when Pin1 expression is inhibited;

FIG. 2A illustrates the result showing that the expression of the NICD1 protein increases in proportion to an expression concentration, when Pin1 is overexpressed in a mouse embryonic fibroblast, an MEF cell, and FIG. 2B illustrates the experimental result of verifying nuclear translocation of the NICD1 protein depending on the Pin1 expression;

FIGS. 3A-3B illustrates the experimental results of verifying the regulation of NICD1 ubiquitination by Pin1, in which FIG. 3A illustrates the result showing that the NICD1 ubiquitination decreases when Pin1 is overexpressed, but increases again due to the treatment of a Pin1 inhibitor, juglone, and FIG. 3B illustrates the result showing that the NICD1 ubiquitination did not decreases when Pin1 mutation takes place;

FIGS. 4A-4G illustrates the experimental results of verifying the inhibitory effect of the juglone on neuronal cell death by Pin1 in an ischemic stroke-like environment, in which FIG. 4A illustrates the expression of Pin1 in the cytoplasm of a neuronal cell in the ischemic stroke-like environment, FIG. 4B illustrates the result showing that the expression of Pin1 protein decreases due to juglone treatment, FIG. 4C illustrates the result showing that the neuronal cell death decreases due to the juglone treatment, FIGS. 4D and 4E illustrate the results showing that the neuronal cell death decreases due to the juglone treatment on a molecular level, and FIGS. 4F and 4G illustrate the results showing that the NICD1 protein decreases due to the juglone treatment;

FIGS. 5A-5D illustrates the experimental results of verifying that the neuronal cell death is inhibited by the treatment of Pin1 inhibitors, juglone, PiB, and DTM, in the ischemic stroke-like environment, in which FIGS. 5A and 5B illustrate the results showing the apoptosis inhibitory effect of the treatment of juglone, PiB, and DTM after apoptosis is induced by exposing SH-SY5Y cells to each of the glucose deprivation (GD) and oxygen and glucose deprivation (OGD) environments for 6 hours, and FIGS. 5C and 5D illustrate the results showing the apoptosis inhibitory effect after the SH-SY5Y cells are exposed to each of the GD and OGD environments for 12 hours;

FIGS. 6A-6E illustrates the experimental results of verifying the importance of Pin1 expression in the occurrence of ischemic stroke using a normal control group mouse (Pin1(+/+)), a heterozygote of Pin1 mouse (Pin1(+/−)), and a Pin1 knockout mouse (Pin1−/−)), in which FIGS. 6A and 6B illustrate the results of the comparison in degree of generation of ischemia in the brain of a mouse by the Pin1 expression, FIG. 6C illustrates the result showing that the neurological deficits decrease when Pin1 is deleted, and FIGS. 6D and 6E illustrate the results showing that the expression of NICD1 protein decreases and the expression of a ubiquitinating enzyme, FBW7 protein, increases by a decrease in expression level of Pin1;

FIGS. 7A-7E illustrates the experimental results of verifying the effect of inhibiting the occurrence of stroke by the administration of the Pin1 inhibitor, juglone, in a middle cerebral artery occlusion (MCAO) animal model, in which FIGS. 7A and 7B illustrate the results showing that the generation of ischemia and neurological deficits decrease in the brain of a mouse due to the juglone administration, FIGS. 7C and 7D illustrate the results showing that the expressions of the NICD1 and Pin1 proteins decrease and the expression of FBW7 protein increases due to the juglone administration, and FIG. 7E is an image showing that the expression of the Pin1 protein increases in the brain of a stroke patient; and

FIGS. 8A-8D illustrates the result showing the neuroprotective effect of the administration of the Pin1 inhibitor, juglone, after ischemic stroke is caused using an MCAO animal model, in which FIGS. 8A and 8B illustrate the results showing that the generation of ischemia and neurological deficits decrease in the brain of a mouse due to the juglone administration, and FIGS. 8C and 8D illustrate the results showing that the expressions of the NICD1 and Pin1 proteins decrease, and the expression of the FBW7 protein increases due to the juglone administration.

### DETAILED DESCRIPTION OR EXEMPLARY EMBODIMENTS

The present invention provides a pharmaceutical composition for preventing or treating stroke, which contains a Pin1 inhibitor as an active ingredient.

In the present invention, the stroke may be ischemic stroke, but the present invention is not limited thereto.

The term “prevention” used herein means all behaviors of inhibiting or delaying the occurrence of stroke by the administration of the pharmaceutical composition according to the present invention.

The term “treatment” used herein means all behaviors of alleviating or beneficially changing symptoms caused by stroke due to the administration of the pharmaceutical composition according to the present invention.

In the present invention, the inhibitor has a function of inhibiting the expression or activity of Pin1, and may be, but is not limited to, at least one selected from the group consisting of 5-hydroxy-1,4-naphthoquinone (juglone), diethyl-1,3,6,8-tetrahydro-1,3,6,8-tetraoxobenzo phenanthroline-2,7-diacetate (PiB), and dipentamethylene thiuram monosulfide (DTM).

Meanwhile, the compounds may be prepared by a known chemical synthesis method, or may be used by purchasing a commercially available reagent.

In the present invention, Pin1 was identified as an activity regulatory factor of Notch1, which is a signaling pathway activated when apoptosis is induced by stroke, and it was confirmed that Pin1 activates Notch1 and increases stability, and thus is involved in the induction of neuronal cell death.

In one example embodiment of the present invention, to confirm the correlation between Notch1 and Pin1 that regulates the signaling activity of Notch1, Pin1 protein was overexpressed in a human neuroblastoma cell line, SH-SY5Y, using Pin1 cDNA, and then a change in expression of NICD1 protein, which is an active form of Notch1, was observed. As a result, it was confirmed that the expression of the NICD1 protein increases when Pin1 is overexpressed. Afterward, by the result of the treatment of DAPT, which is an inhibitor of γ-secretase cleaving NICD1, in combination with the Pin1 overexpression, NICD1 did not increase. According to the result, it was confirmed that Pin1 overexpression has an influence on γ-secretase-mediated Notch1 signaling activity and NICD1 stability. Also, the activation of Notch1 signaling by Pin1 was reconfirmed by observing siSNA concentration-dependent reduction in expression of the NICD1 protein when the expression of Pin1 was inhibited by treating cells with Pin1 siRNA (see Example 2-1). Also, it was observed that Pin1 also regulated a nuclear translocation efficiency of NICD1, and thereby confirmed that Pin1 activates and stabilizes Notch1 (see Example 2-2).

In another example embodiment of the present invention, in order to verify that Pin1 also has an influence on proteolytic degradation by ubiquitination of the NICD1 protein, a change in ubiquitination occurring in the proteolytic degradation of NICD1 was observed. As a result, when Pin1 was overexpressed, the ubiquitination of NICD1 was reduced, and when the Pin1 inhibitor, juglone, was treated or mutation took place in the Pin1 protein, the ubiquitination of NICD1 was not reduced. Therefore, it was confirmed that Pin1 inhibits the proteolytic degradation by the ubiquitination of NICD1, thereby increasing stability (see Example 3).

In still another example embodiment of the present invention, it was verified that the neuronal cell death is inhibited by the Pin1 inhibitor in the ischemic stroke-like environment. Primary neuronal cells were cultured in a glucose, serum-free medium under glucose deprivation (GD), which is a 5% oxygen-supplying environment, and oxygen and glucose deprivation (OGD), which is an environment from which glucose and serum are removed and to which 1% oxygen is supplied, the expression of Pin1 protein in the neuronal cells increased, and then neuronal cell death was induced in both of the environments. However, when the cells were treated with juglone under the same environment, the expression of the Pin1 protein and the neuronal cell death decreased. Also, it was confirmed that production of the NICD1 protein increased was reduced by the juglone treatment, and the amount of an apoptosis signal protein, cleaved caspase-3, was also reduced (see Example 4). Afterward, even when an SH-SY5Y cell line was treated with Pin1 inhibitors other than juglone, such as PiB and DTM, in the same ischemic stroke-like environment, the apoptosis inhibitory effect was confirmed (see Example 5).

Also, in the present invention, it was confirmed that in the MCAO animal model, the Pin1 inhibitor, juglone, had an inhibitory effect on the occurrence of stroke and a neuroprotective effect to inhibit the neuronal cell death caused by ischemic stroke.

In one example embodiment of the present invention, in order to verify the influence of Pin1 on the occurrence of ischemic stroke before the effects of the Pin1 inhibitor were confirmed, ischemic stroke was triggered through an MCAO operation in a normal control group mouse, a Pin1-heterozygous mouse, and a Pin1-knockout mouse. As a result, it was confirmed that, compared to the control group, the generation of ischemia and neurological deficits, which are shown as the result of ischemic stroke, decreased in the heterozygote and knockout mice, and the expression of the NICD1 protein decreased, but the expression of a ubiquitinating enzyme, FBW7 protein, increased in the brain cells due to the decrease in expression of the Pin1 protein (see Example 6).

In another example embodiment of the present invention, ischemic stroke was triggered in mice by intraperitoneal administration of the juglone and an MCAO operation, and then the resultant mice were compared with the control group to which juglone was not administered. As a result, compared to the control group, the generation of ischemia and the neurological deficits decreased, and the expressions of NICD1 and Pin1 proteins decreased but the expression of the FBW7 protein increased in the brain cells. In addition, the increase in the expression of the Pin1 protein in tissues of a human patient with ischemic stroke was confirmed by Pin1 staining through immunohistochemistry (see Example 7).

In still another example embodiment of the present invention, an MCAO operation was conducted on mice to trigger ischemic stroke, thereby inducing neuronal cell death, and then it was verified that the neuronal cell death was inhibited by the administration of the Pin1 inhibitor, juglone. As a result, compared to the control group to which the juglone was not administered, the generation of ischemia and the neurological deficits decreased, and the expressions of the NICD1 and Pin1 proteins in the brain cells decreased, however, the expression of the FBW7 protein increased. Therefore, it was confirmed that the Pin1 inhibitor, juglone, has a neuroprotective effect even after ischemic stroke is triggered (see Example 8).

The pharmaceutical composition according to the present invention may contain a pharmaceutically acceptable carrier, in addition to the active ingredient. Here, the pharmaceutically acceptable carrier is one that is conventionally used in preparation, which may include, but is not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. Also, in addition to the above components, the pharmaceutical composition of the present invention may further contain a lubricant, a wetting agent, a sweetener, a flavoring agent, an emulsifier, a suspending agent, and a preservative.

According to a desired method, the pharmaceutical composition of the present invention may be administered orally or parenterally (e.g., intravenously, subcutaneously, intraperitoneally, or locally), and a dosage may be changed depending on the condition and body weight of a patient, severity of a disease, a type of a drug, an administration route, and duration, but may be suitably determined by those of ordinary skill in the art.

The composition according to the present invention is administered at a pharmaceutically effective amount. As used herein, the “pharmaceutically effective amount” means an amount sufficient to treat a disease at a reasonable benefit/risk ratio applicable for medical treatment, and an effective capacity level may be determined by parameters including a type of a patient disease, severity, drug activity, sensitivity to a drug, administration time, an administration pathway, a release rate, duration of treatment, and co-used drugs, as well as other parameters well known in the medical field. The composition according to the present invention may be administered alone as an individual therapeutic agent or in combination with a different therapeutic agent, administered sequentially or simultaneously with a conventional therapeutic agent, or administered once or multiple times. Taking all of the above parameters into consideration, it is important to administer such an amount as to obtain the maximum effect with the minimal amount without side effects, and the amount may be easily determined by those of ordinary skill in the art.

Specifically, the effective amount of the compound according to the present invention may vary depending on the age, sex and body weight of a patient, and generally the compound may be administered at 0.001 to 150 mg, and preferably, 0.01 to 100 mg per 1 kg of body weight, daily or every other day, or once to three times a day. However, the effective amount may vary depending on the administration route, severity of obesity, sex, body weight, and age, and therefore the scope of the present invention is not limited to the dosage, regardless of the method.

Hereinafter, exemplary examples will be provided to help in understanding of the present invention. However, the following examples are merely provided to facilitate understanding of the present invention, and the scope of the present invention is not limited to the following examples.

### EXAMPLES

### Example 1

### Preparation for Experiment Materials

1-1. Culture of Cell Lines and Preparation of Reagents

In Examples according to the present invention, a human neuroblastoma cell line, SH-SY5Y, and a mouse embryonic fibroblast cell line, an MEF cell, were used. All of the cells were cultured in DMEM (Sigma) media containing 10% fetal bovine serum (Gibco, Welgene, Hyclone) and 1% penicillin/streptomycin.

A Pin1 inhibitor, 5-hydroxy-1,4-naphthoquinone (juglone), which was used in the following examples, was prepared to be dissolved in dimethylsulfoxide (DMSO) to have a final concentration of 1-5 μM.

1-2. Western Blot Analysis

A sample was prepared by isolating a protein from cells, and analyzed by 10% sodium dodecyl sulfate (SDS)-polyacrylamide gel electrophoresis using Tris-glycine running buffer. The gel was removed after the electrophoresis, and a transferring process was performed using a transfer buffer containing 0.025 mol/L Tris base, 0.15 mol/L glycine, and 10% (v/v) methanol at 15 V for 1.5 hours to transfer proteins onto a nitrocellulose membrane along an electrical flow. The protein-binding membrane was immersed in blocking buffer (5% non-fat milk in 20 mM Tris-HCl, pH 7.5, 137 mM NaCl, 0.2% Tween-20), and reacted at room temperature for 1 hour. Afterward, the membrane was reacted overnight with primary antibodies, such as Pin1 (R&D, Epitomics), NICD1 (Upstate), MYC (Roche), FBW7 (Bioss), or Actin (Sigma), which specifically bound to each protein whose expression level was to be confirmed, at 4° C. The next day, the membrane was washed with Tris-buffered saline-T (20 mM Tris-HCL, pH 7.5, 137 mM NaCl, 0.2% Tween-20) three times each for 10 minutes. In addition, the membrane was reacted with secondary antibodies at room temperature for 1 hour, washed again three times, and then scanned using an Odyssey® Infrared Imaging System (LI-COR Biosciences) or ECL-plus system to analyze the expression level of each protein.

1-3. Immunocytochemistry

Pin1-free mouse embryonic fibroblasts (MEF), that is, MEF Pin1(−/−) cells, were incubated on a cover glass. 1.5 μg of Pin1 cDNA was overexpressed, the equal amount of control cDNA was expressed in MEF Pin1(−/−) and MEF Pin1(+/+) cells, and after 24 hours, the cells were fixed by treating 4% paraformaldehyde in phosphate-buffered saline (PBS; pH 7.4) buffer for 5 minutes. Afterward, the cells were treated with 0.2% Triton-X-100 in PBS buffer for 10 minutes, washed with PBS buffer three times each for 10 minutes, and treated with 5% bovine serum albumin (BSA) in PBS buffer for 1 hour. Primary antibodies of Pin1 and NICD1, specifically binding to proteins of interest as primary antibodies were mixed with 1% BSA in PBS buffer at a ratio of 1:50, and the cells were treated with the resultant mixture and reacted overnight at 4° C., washed again with PBS buffer three times each for 10 minutes, and treated with fluorescein isothiocyanate (FITC)-conjugated IgG specifically binding to the primary antibodies at room temperature for 1 hour. Subsequently, the cells were washed with PBS buffer three times. After 10 μl of a mounting solution mixed with DAPI reagent for nuclear staining was applied to a slide glass, the side glass was covered with a cover glass to observe a fluorescent image by confocal microscope.

1-4. Construction of MCAO Animal Models

A mouse was anesthetized and an epidermis around the neck was incised. The left common carotid artery linked to the left hemisphere was ligated with a surgical suture, and then the external carotid artery was incised to insert a filament thereinto. The filament was pushed into the middle cerebral artery through the internal carotid artery to locally clog the middle cerebral artery, thereby interrupting blood supply to construct an ischemic animal model. After cerebral infarction was induced in the animal model for 1 hour, the filament was removed from the blood vessel (artery), and then the blood was supplied again to the blood vessel, followed by suturing.

### Example 2

### Verification of Regulation of Notch1 Signaling Activity by Pin1

2-1. Confirmation of Regulation of Notch1 Signaling Activity by Pin1

First, to verify the correlation between the Notch1 signaling activity and Pin1, Pin1 protein was overexpressed with Pin1 cDNA in a human neuroblastoma cell line, SH-SY5Y, and a change in expression of a Notch1 signaling-activation molecule, NICD1 protein, was analyzed by western blotting (WB).

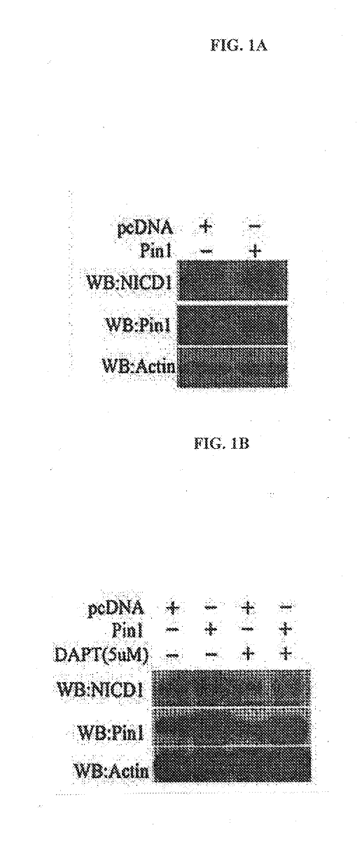

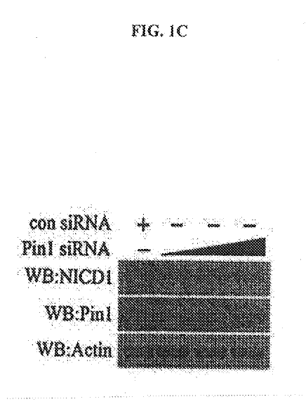

As a result, as illustrated in FIG. 1A, when Pin1 was overexpressed, compared to a control group (pcDNA) containing plasmid cDNA that is not involved in gene expression, the expression of NICD1 protein increased. This shows that Notch1, which is a membrane protein embedded in a cell membrane, is cleaved by γ-secretase to become an activated form, NICD1, and the inhibition of the production of NICD1, which was mediated by a γ-secretase inhibitor, N-[N-(3,5-difluorophenacetyl)-1-alanyl]-S-phenylglycine t-butyl ester (DAPT), was not partially attained due to the overexpression of the Pin1 protein. To verify this, as in FIG. 1B, 5 μM of the γ-secretase inhibitor, DAPT, was treated in combination after the overexpression of Pin1, and thereby the increase in NICD1 was not found. In other words, it was confirmed that the overexpression of the Pin1 protein has an influence on the γ-secretase-mediated Notch1 signaling activity and the NICD1 stability. In contrast, Pin1 expression was inhibited with siRNA specific to Pin1 capable of inhibiting the expression of a protein by specifically degrading mRNA of Pin1. As a result, as illustrated in FIG. 1C, it was confirmed that, compared to the control group treated with control siRNA, the production of NICD1 protein decreased in proportion to the concentration of treated Pin1 siRNA.

2-2. Confirmation of Regulation of Nuclear Translocation of NICD1 by Pin1 Protein

It is reported that the active form of Notch1, NICD1 protein, is translocated into a nucleus and interacts with transcriptional co-factors serving to regulate gene expression. Therefore, it was verified that the Pin protein has an influence on production and nuclear translocation of NICD1.

After the Pin1 protein was expressed again with Pin1 cDNA in Pin1-free mouse embryonic fibroblasts (MEFs), the change of NICD1 protein was analyzed by western blotting (WB). In addition, immunocytochemistry and confocal microscope were carried out to visualize the nuclear translocation of NICD1 with a fluorescent image.

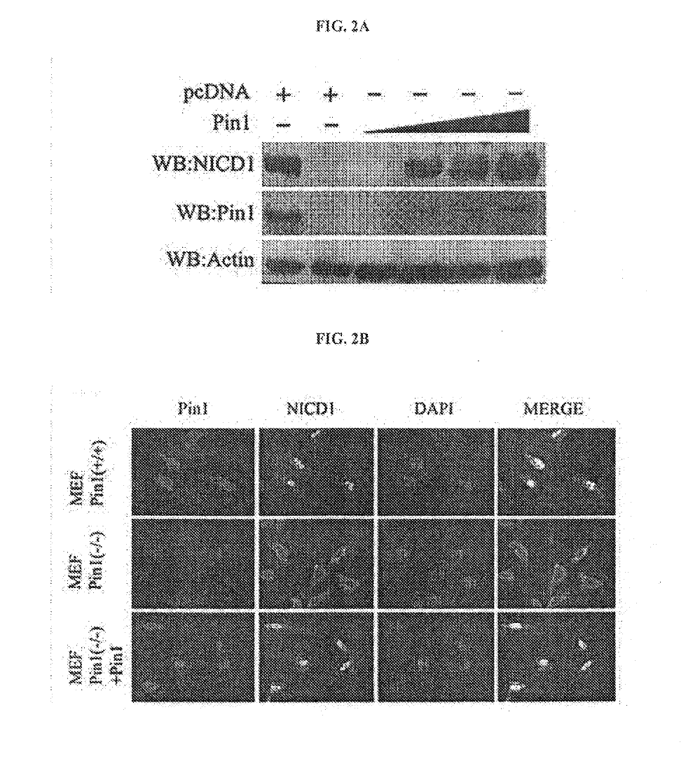

As a result, as illustrated in FIG. 2A, it was found that in Pin1-free intact MEF cells (MEF Pin1(−/−)), the NICD1 protein was rarely expressed, whereas in Pin1 protein-reexpressed MEF cells (MEF Pin1(+/+)), the NICD1 protein was reproduced Pin1 cDNA-concentration dependently. Also, as the result of the immunocytochemistry, as illustrated in FIG. 2B, when the nuclei of the cells were stained with DAPI and then compared with an overlap image (MERGE), the MEF Pin1(−/−) cells had a very low NICD1 level in the nucleus and were distributed in the cytoplasm, except the nucleus, whereas when Pin1 was added again to the Pin1-free intact MEF cells (MEF Pin1(−/−)+Pin1), the NICD1 protein level in the nucleus was similar to that in the MEF Pin1(+/+) cells.

### Example 3

### Verification of Regulation of Ubiquitination of NICD1 by Pin1

The NICD1 protein is known to be degraded in the nucleus by F-box and WD repeats domain-containing 7 (FBW7) in accordance with a proteasome-dependent pathway. However, Example 2 showed that Pin1 regulates NICD1 stability, and it was verified that Pin1 has an influence on proteolytic degradation by the ubiquitination of the NICD1 protein.

MYC-tagged NICD1, HA-tagged ubiquitin (Ub), and Pin1 were expressed in SH-SY5Y cell lines, and treated with MG132 to inhibit a proteolytic activity of a 26S proteasome complex. Afterward, HA and FBW7-specific antibodies were used to analyze a degree of NICD1 ubiquitination by co-immunoprecipitation (IP) and western blotting (WB).

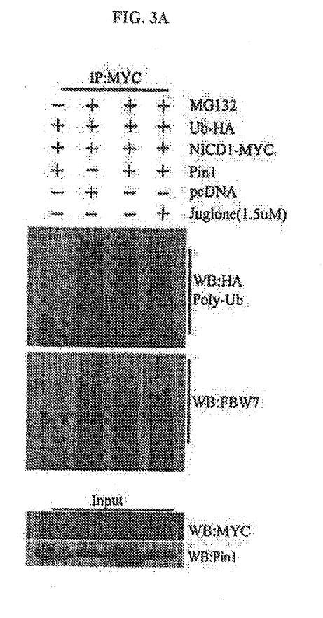

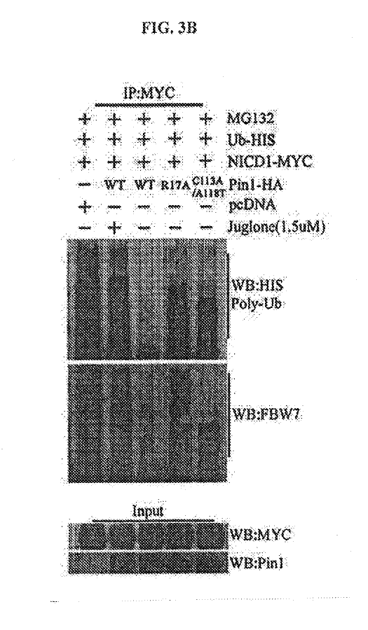

As a result, as illustrated in FIG. 3A, when Pin1 was overexpressed, compared to a control group using plasmid cDNA (pcDNA), the NICD1 ubiquitination was reduced. However, when a Pin1 inhibitor, juglone, was treated at 1.5 μM, the NICD1 ubiquitination was increased again.

Subsequently, mutants (R17A, C113A/A118T) of Pin1 protein were formed by substituting a part of the constitutive amino acids of WW domain binding to a substrate and PPIase domain having an isomerase activity among the domains constituting the Pin1 protein with another amino acid, and then the same experiment as described above was carried out.

As a result, as illustrated in FIG. 3B, when the normal Pin1 was overexpressed, the NICD1 ubiquitination was reduced, whereas when the mutant Pin1 was overexpressed, NICD1 did not decrease. Consequently, it was confirmed that Pin1 enhances the stability of the NICD1 protein by inhibiting the proteolytic degradation by reducing the ubiquitination of NICD1.

### Example 4

### Verification of Neuroprotective Effect of Juglone in Ischemic Stroke-Like Environment

According to Examples 2 and 3, it was confirmed that Pin1 has an influence on Notch1 signaling involved in neuronal cell death, and here, when the neuronal cell death is induced under an ischemic stroke-like condition, a change in Pin1 expression and the inhibition of the neuronal cell death by the treatment of the Pin1 inhibitor, juglone, were verified.

Primary neuronal cells were cultured in a glucose and serum-free medium in a 5% or 1% oxygen incubator for 9, 12 and 24 hours to create an ischemic stroke-like environment on a cellular level, thereby inducing the neuronal cell death. Afterward, an expression level of the Pin1 protein was analyzed by immunocytochemistry and western blotting.

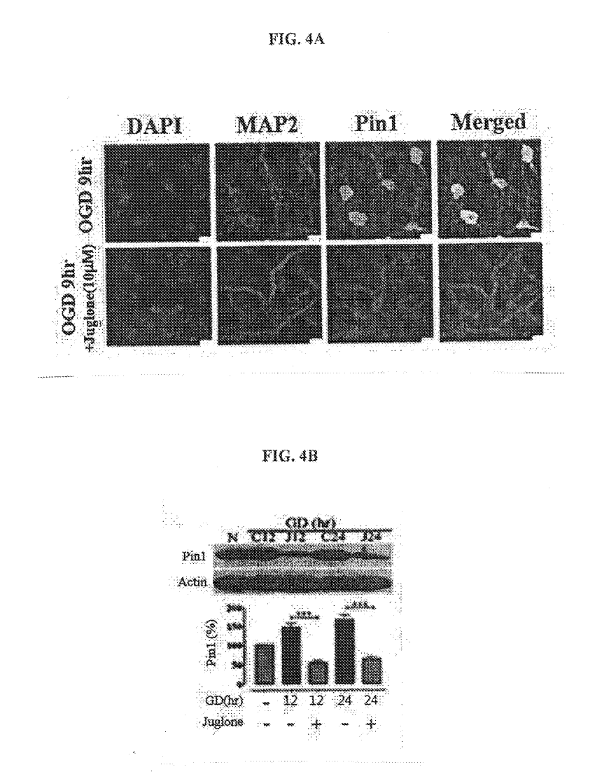

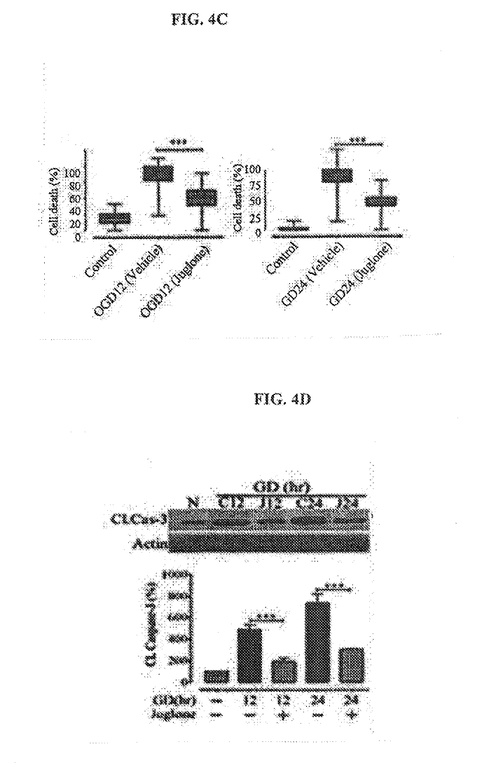

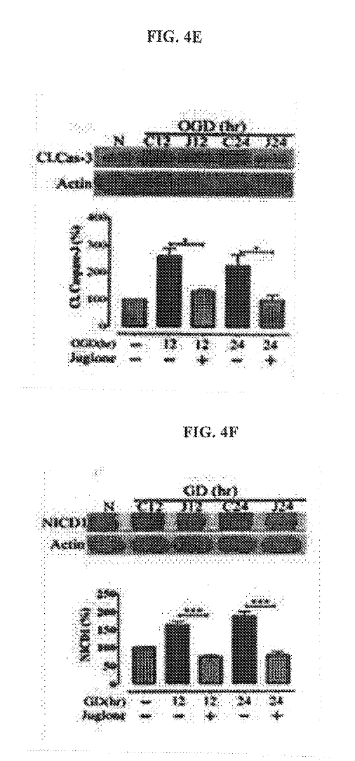

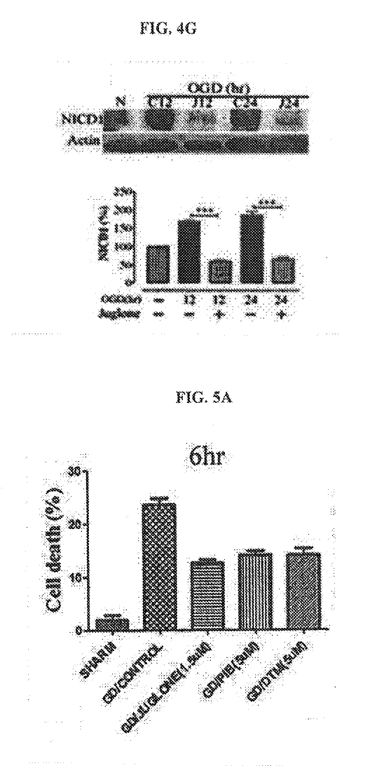

As a result, as illustrated in FIG. 4A, when neuronal cells were exposed under an oxygen and glucose deprivation (OGD) condition including a glucose and serum-free medium and a 1% oxygen incubator, the increase in Pin1 protein was confirmed by the expressions of a neuronal cell marker, MAP2, and green fluorescence. Also, by observing the image merged with nuclear staining with DAPI, strong expression of Pin1 was detected in the cytoplasm. Also, as in FIG. 4B, even when neuronal cells were cultured under a glucose deprivation (GD) condition including culture in a glucose and serum-free medium for 12 hours or 24 hours, the expression of the Pin1 protein increased in all groups. In addition, after the neuronal cells were cultured under the OGD condition for 12 hours, the induction of the neuronal cell death was detected by trypan blue (0.4%) staining, which is shown in FIG. 4C. Meanwhile, it was confirmed that the increases in Pin1 expression and apoptosis shown in FIGS. 4A to 4C were reduced by juglone treatment.

Subsequently, primary neuronal cells were cultured under a GD or OGD condition for 12 hours or 24 hours, and then changes in expressions of caspase-3, which is one of the apoptosis signal proteins, and NICD1 protein were analyzed by western blotting.

As a result, as illustrated in FIGS. 4D and 4E, under both of the culture conditions and over both of the culture periods, the expression of the active form of caspase-3, which is a cleaved caspase-3 (CLCas-3) protein, increased, but was decreased by the juglone treatment. Also, as illustrated in FIGS. 4F and 4G, it was confirmed that the expression of NICD1 protein increased under both of the culture conditions, but was decreased by the juglone treatment. Consequently, it was confirmed that an increase in apoptosis signals caused by the increase in NICD1 in ischemic stroke can be inhibited by the inhibition of Pin1.

### Example 5

### Verification of Neuroprotective Effect of Pin1 Inhibitors Including Juglone

By the method as described in Example 4, SH-SY5Y cells were exposed to OGD or GD conditions to induce apoptosis, and Pin1 inhibitors including juglone were treated to verify a neuroprotective effect.

SH-SY5Y cells were treated with Pin1 inhibitors such as 5-hydroxy-1,4-naphthoquinone (juglone), diethyl-1,3,6,8-tetrahydro-1,3,6,8-tetraoxobenzo phenanthroline-2,7-diacetate (PiB), and dipentamethylene thiuram monosulfide (DTM) at a concentration each of 1.5, 5 or 5 μM, and exposed to a GD or OGD condition for 6 hours or 12 hours to induce apoptosis, and then stained with trypan blue (0.4%) to quantify a degree of apoptosis.

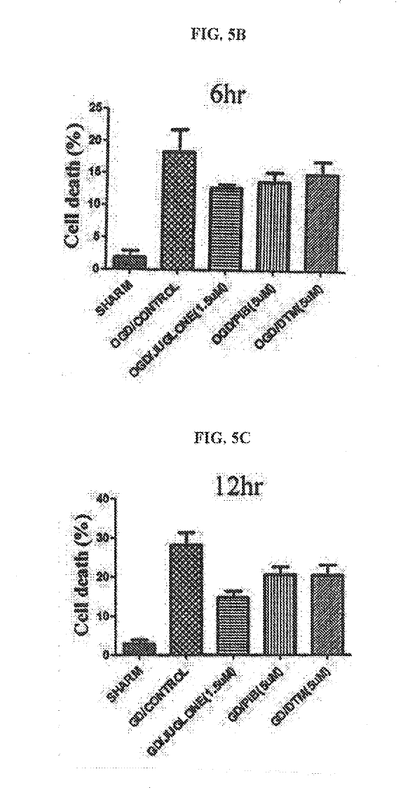

As a result, as illustrated in FIGS. 5A to 5D, when the cells were exposed to an OGD or GD condition for 6 hours or 12 hours to induce apoptosis and then treated with the Pin1 inhibitors, in every group treated with the Pin1 inhibitor, the apoptosis decreased to a greater or less extent. That is, the neuroprotective effects of the Pin1 inhibitors such as juglone, PiB, and DTM were confirmed.

### Example 6

### Verification of Effect of Pin1 in Ischemic Stroke Animal Models

To verify in vivo that Pin1 has an important influence on the real occurrence of ischemic stroke, the following animal test was carried out.

A normal control group mouse (Pin1(+/+)) normally expressing Pin1, a heterozygous mouse (Pin1(+/−)) in which only one of the pair of homologous chromosomes normally expressed Pin1, and a knockout mouse (Pin1(−/−)) that did not express Pin1 at all were prepared, and as described in Example 1-4, ischemic stroke was triggered in the mice by a middle cerebral artery occlusion (MCAO) operation, and then the resultant groups were compared.

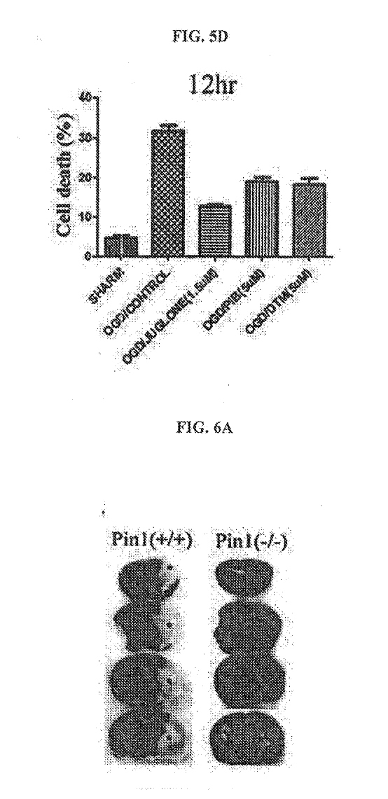

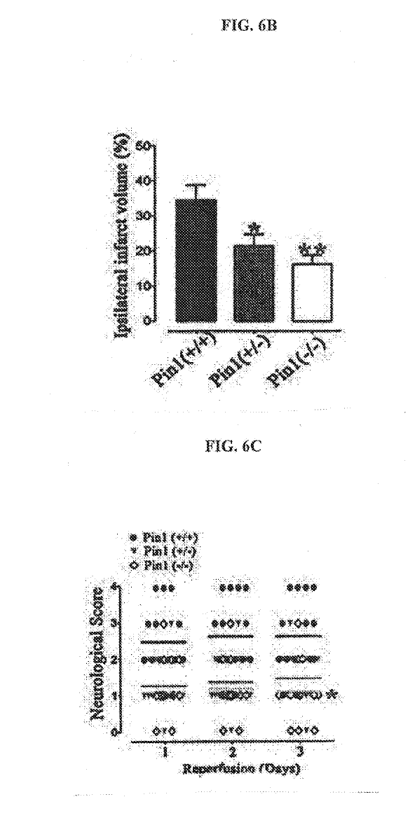

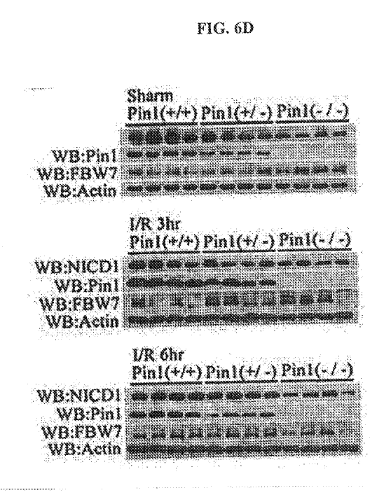

As a result, as illustrated in FIGS. 6A and 6B, the brain was removed and stained with 2,3,5-triphenyltetrazolium chloride (TTC) to estimate the size of cerebral infarction with the naked eye. Upon the comparison in the generation of ischemia between groups, compared to a normal control group, Pin1 heterozygous and knockout mice showed decreasing generation of ischemia. Also, as in FIG. 6C, it was confirmed that, in the knockout mouse, neurological deficits caused by the death of brain neuronal cells due to the triggering of stroke also decreased. Afterward, the removed left hemisphere tissues were analyzed by western blotting (WB) with antibodies specific to each of NICD1, Pin1, and FBW7 to compare the expression of each protein. As a result, as illustrated in FIGS. 6D to 6E, in the Pin1 heterozygous and knockout mice, the expression of the NICD1 protein was decreased by a decrease in expression level of the Pin1 protein, and the expression of the NICD1 ubiquitinating enzyme, FBW7 protein, increased. Consequently, it was confirmed that Pin1 is the very important factor in the occurrence of stroke.

### Example 7

### Verification of Stroke Inhibitory Effect of Juglone in MCAO Animal Model

Example 6 showed that Pin1 is the very important factor in the occurrence of stroke, and thus, a stroke inhibitory effect of the Pin1 inhibitor, juglone, in the middle cerebral artery occlusion (MCAO) animal model was verified.

Ischemic stroke was trigged in the mouse by intraperitoneally administering the juglone (3 mg/kg) twice and conducting an MCAO operation, and then the resultant mouse was compared with a control group mouse to which the juglone was not administered.

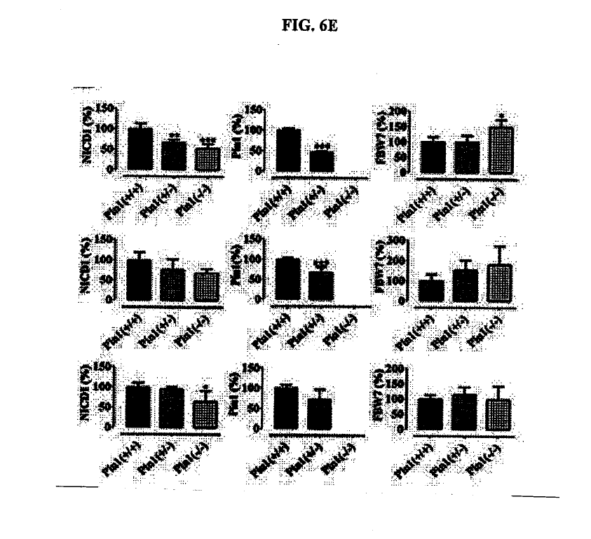

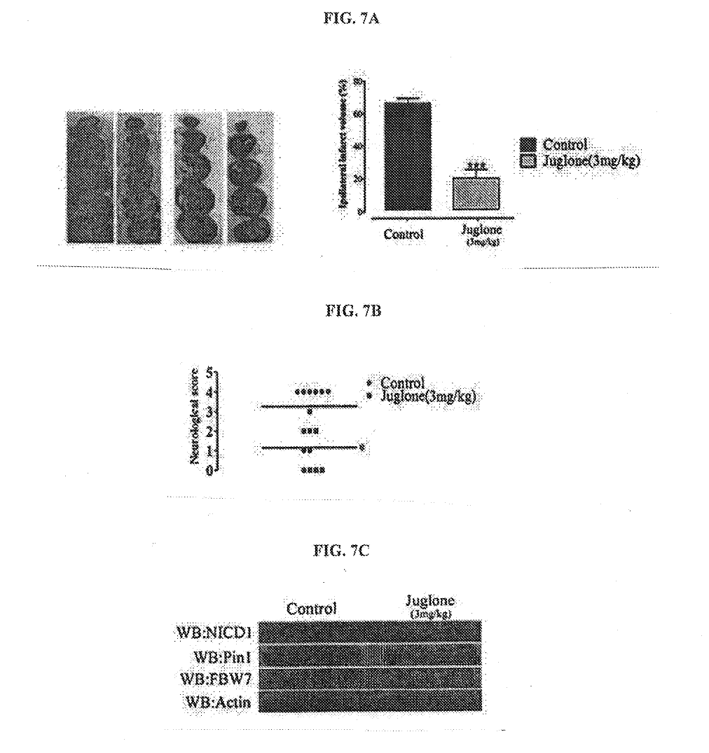

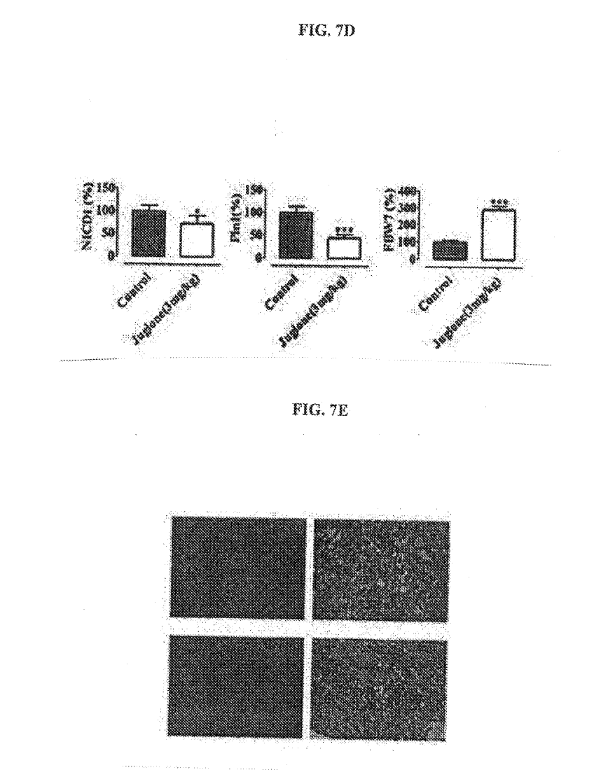

As a result, compared to the control group, in the juglone-administered mouse, the generation of ischemia decreased as in FIG. 7A, and the neurological deficits also decreased as in FIG. 7B. In addition, the left hemisphere of the stroke-triggered mouse was analyzed by western blotting to compare the expressions of NICD1, Pin1 and FBW7 proteins. As a result, as illustrated in FIGS. 7C and 7D, compared to the control group, the expressions of the NICD1 and Pin1 proteins decreased, but the expression of the FBW7 protein increased in the juglone-administered mouse. Also, FIG. 7E showed that, compared to a normal person, the expression of the Pin1 protein also increased in the brain tissue of a stroke patient, which was visualized by staining the Pin1 protein through immunohistochemistry.

### Example 8

### Verification of Neuroprotective Effect by Pin1 Inhibition after Triggering of Ischemic Stroke

Afterward, it was verified that the neuronal cell death was inhibited by the treatment of the Pin1 inhibitor, juglone, when the neuronal cell death was induced by the triggering of ischemic stroke.

An MCAO operation was conducted on a mouse as described in Example 1-4 to trigger ischemic stroke, and then juglone (3 mg/kg) was administered once to the mouse by intravenous injection. Afterward, the resultant mouse was compared with the control group mouse to which the juglone was not administered.

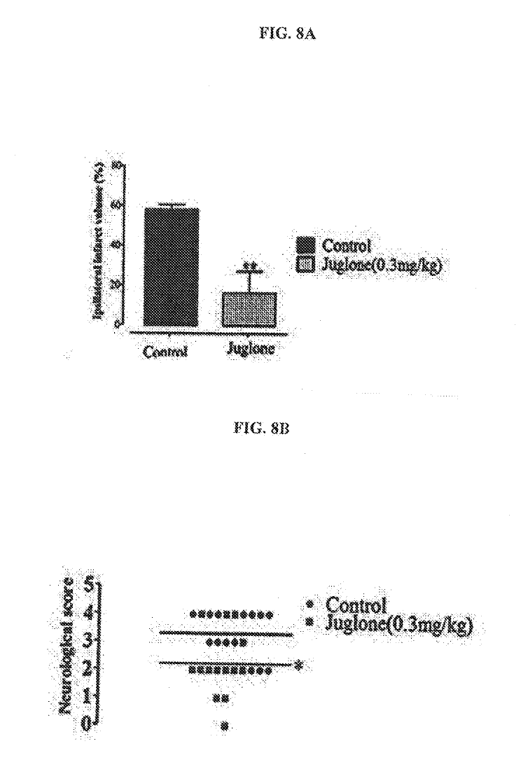

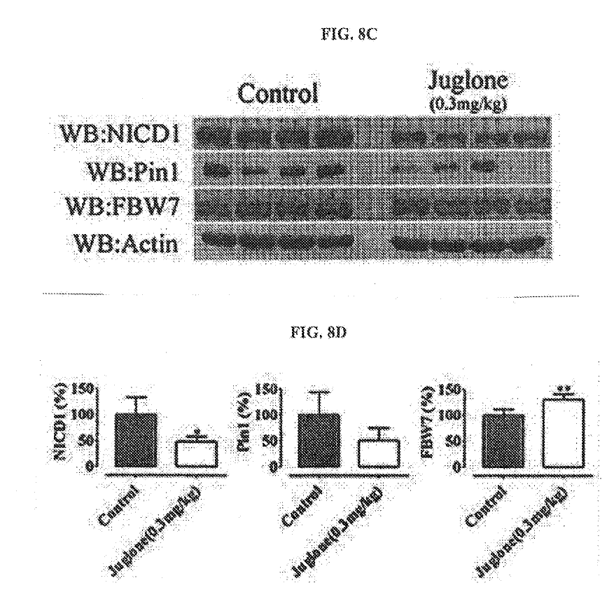

As a result, as illustrated in FIGS. 8A and 8B, it was confirmed that, compared to the control group, the generation of ischemia and the neurological deficits decreased in the juglone-administered mouse. Also, upon the analysis of the expressions of NICD1, Pin1 and FBW7 proteins by western blotting as described in Example 6 and 7, as in FIGS. 8C and 8D, compared to the control group, in the juglone-administered mouse, the expressions of NICD1 and Pin1 proteins decreased, and the expression of FBW7 protein increased. Consequently, it was confirmed that the Pin1 inhibitor, juglone, has a neuroprotective effect even after ischemic stroke is triggered.

It would be understood by those of ordinary skill in the art that the above descriptions of the present invention are exemplary, and the example embodiments disclosed herein can be easily modified into other specific forms without changing the technical spirit or essential features of the present invention. Therefore, it should be interpreted that the example embodiments described above are exemplary in all aspects, and are not limitative.

What is claimed is:

1. A method of preventing or treating stroke, comprising:
administering a composition comprising a peptidyl-prolyl cis-trans isomerase NIMA-interacting 1 (Pin1) inhibitor as an active ingredient to a subject.

2. The method of claim 1, wherein the stroke is ischemic stroke.

3. The method of claim 1, wherein the inhibitor is at least one selected from the group consisting of 5-hydroxy-1,4-naphthoquinone (juglone), diethyl-1,3,6,8-tetrahydro-1,3,6,8-tetraoxobenzo phenanthroline-2,7-diacetate (PiB), and dipentamethylene thiuram monosulfide (DTM).

4. The method of claim 1, wherein the composition inhibits the expression or activity of Pin1.

5. The method of claim 1, wherein the composition has a neuroprotective effect by inhibiting the activity of Notch1.

6. The method of claim 1, wherein the composition further comprises:
a pharmaceutically acceptable carrier or additive.
